(19) United States
(12) Patent Application Publication
(10) Pub. No.: US 2026/0141815 A1
(43) Pub. Date: May 21, 2026

(54) METHOD FOR AGENT EMPATHETIC RESPONSE BASED ON BRAIN-MACHINE COUPLING UNDER GUIDANCE OF LEARNERS EMOTION

(71) Applicant: Zhejiang Normal University, Jinhua (CN)

(72) Inventors: Zhongmei HAN, Jinhua (CN); Wanpin ZHANG, Jinhua (CN); Changqin HUANG, Jinhua (CN); Xiaobo LI, Jinhua (CN)

(73) Assignee: Zhejiang Normal University, Jinhua, ZJ (CN)

(21) Appl. No.: 19/446,835

(22) Filed: Jan. 12, 2026

(30) Foreign Application Priority Data
Mar. 4, 2025 (CN) 202510247708.5

(51) Int. Cl. G09B 5/00; A61B 5/16; A61B 5/372; A61B 5/378; G06F 3/01; G06F 40/284; G06F 40/30; G06F 40/40; G06V 10/40; G06V 10/82; G06V 40/16; G10L 25/63

(52) U.S. Cl. CPC G09B 5/00; A61B 5/165; A61B 5/372; A61B 5/378; G06F 3/015; G06F 40/284; G06F 40/30; G06F 40/40; G06V 10/40; G06V 10/82; G06V 40/168; G06V 40/174; G10L 25/63; G06F 2203/011

(57) ABSTRACT

Disclosed is a method for agent empathetic response based on brain-machine coupling under a guidance of learner's emotion comprising steps of: collecting multimodal affective data of learners directed to an agent empathetic response; performing an emotion analysis on the collected multimodal affective data of the learner under a brain-machine coupling by employing an affective state discrimination method of the multimodal data; and designing a multimodal empathetic response generation framework based on results of emotion recognition and a dialogue content, constructing an empathetic response model based on a multimodal representation, and performing a joint learning training of the empathetic response model. An agent empathetic response model for a multi-agent system based on a LLM is formed under the guidance of the learner's emotion. The learner's empathetic ability is enhanced by accurately identifying the learner's emotional state and dynamically adjusting the agent's dialogue content based on the emotional state.

### TECHNICAL FIELD

The present disclosure pertains to the cross-integration technology field of artificial intelligence and brain science, particularly to a method for agent empathetic response based on brain-machine coupling under a guidance of a learner's emotion.

### BACKGROUND

In the contemporary educational environment, learners are required to master not only subject knowledge but also social-emotional competence and empathetic abilities, to achieve optimal outcomes in increasingly complex learning tasks. Empathy, defined as a capacity for individuals to comprehend, perceive, and appropriately respond to the emotions of others within social interactions, constitutes an integral component of collaborative learning. Research indicates that empathy effectively promotes trust, cooperation, and mutual understanding among individuals, thereby enhancing the depth of thinking and capacity for teamwork of learners. Consequently, cultivating the empathetic abilities of learners becomes a pivotal factor in improving the learning effect.

Nevertheless, the current emphasis in human-computer collaborative education remains predominantly on knowledge transfer, with insufficient attention paid to fostering social-emotional competencies for learners, particularly empathy abilities. Although conventional agents are capable of performing certain emotion recognition and providing feedback during interactions with learners, their capabilities are typically confined to a single modality of emotional expression and lack depth in emotional comprehension. More critically, existing agents frequently prove inadequate in accurately discerning learner's emotional states across diverse situations. Consequently, they fail to generate appropriate empathetic feedback based on the emotional states, resulting in failure to effectively promote learner's empathy within collaborative learning environments. Furthermore, advancements in artificial intelligence and brain-machine interface technology enable agents to perceive and respond to multimodal emotional information, including visual, auditory, and even physiological signals, when interacting with learners. While this evolution presents a promising new direction for designing empathetic agents, the question of how to effectively combine these technologies to generate dialogue content that conforms to learner's emotional states is still a difficult point in current technology.

### SUMMARY

Aiming at the above problems of the prior art, the present disclosure proposes a method for agent empathetic response based on brain-machine coupling under a guidance of a learner's emotion, enabling accurate identification of the learner's emotional state and dynamic adjustment of the agent's dialogue content based on this emotional state. Consequently, it effectively enhances the learner's emotional cognitive abilities during collaborative learning and promotes the development of social interaction skills and emotional intelligence.

In order to achieve the above objective, the present disclosure proposes a method for agent empathetic response based on brain-machine coupling under a guidance of a learner's emotion, and the method includes the following steps:
S1, collecting multimodal affective data of learners directed to an agent empathetic response, in which the collected multimodal affective data includes learner brain data directed to emotional empathy and dialogue data for agent collaboration;
S2, performing an emotion analysis on the collected multimodal affective data of the learner under a brain-machine coupling by employing an affective state discrimination method of the multimodal data, specifically the emotion analysis is performed from three parts: a facial emotion analysis of the learner based on the brain-machine coupled learning method, an emotion recognition of a learner's dialogue text based on a second-order interactive attention mechanism, and an emotional state discrimination of the learner based on a fusion of brain-vision-language multimodal data; and
S3, designing a multimodal empathetic response generation framework based on results of emotion recognition and a dialogue content, then constructing an empathetic response model based on a multimodal representation, and performing a joint learning training of the empathetic response model, thereby forming an agent empathetic response model for a multi-agent system based on a large language model (LLM) under the guidance of the learner's emotion.

In some embodiments, in S1, the specific steps of learner brain data collection directed to emotional empathy are as follows:
S11, collecting the learner's brain data in a laboratory environment, allowing the learner to view different emotional images from a data set to acquire electroencephalogram (EEG) signals; and
S12, removing invalid fragments and artifacts from the acquired EEG signals, and filtering the signals within a frequency band of 1 Hz-75 Hz using a Butterworth filter.

In some embodiments, in S1, the specific steps of dialogue data collection for agent collaboration are as follows: after completing a phased classroom learning task, performing a self-perception of a learning content and a learning emotion by the learner, reporting this to the agent, generating a dialogue and a communication with the agent, and recording a context data set of the dialogue as J=[F₁, . . . , Fᵢ, . . . , F_{N}], recording a dialogue situation as

$$Q = [ {w}_{1}^{q} , {w}_{2}^{q} , … , {w}_{n}^{q} ] ,$$

and enabling the agent model to understand an emotion A within the dialogue; where,

$${F}_{i} = [ {w}_{1}^{i} , {w}_{2}^{i} , … , {w}_{{n}_{i}}^{i} ]$$

denotes an i^{th} utterance, containing nᵢ words, and the situation Q denotes a situation description composed of n words, where w^{q} is situation background information.

In some embodiments, in S2, the specific steps of the facial emotion analysis of the learner based on the brain-machine coupled learning method are as follows:
S211, performing a preliminary feature extraction on facial expression images and EEG signals by using an EEGNet and a Convolution Neural Network (CNNNet);
wherein for facial expression images in a visual domain, an improved CNNNet is employed to extract preliminary features, with a preliminary representation in the visual domain denoted as \hat{V}:

$$\hat{V} = 𝒢 V ( V ) ;$$

where [symbol] V is the improved CNNNet, V is a given visual image, and \hat{V} is the preliminary representation of the visual domain;
for the EEG signal, a feature extractor of the EEGNet is employed for a preliminary feature extraction, with a preliminary representation in a cognitive domain denoted as \hat{R}:

$$\hat{R} = 𝒢 R ( R ) ;$$

where [symbol] R is a compact convolutional neural network designed by EEGNet for an EEG brain-machine interface, R is a given EEG signal, and \hat{R} is the preliminary representation of the cognitive domain;
S212, constructing a cognitive-visual learning framework, projecting the acquired preliminary representation to a common channel and a private channel, and encoding the common channel and the private channel representations;
where the common channel employs an encoding function G(V, R)(⋅) with shared parameters to learn and capture common information between the cognitive and visual domains, after training, the common channel is configured to extract the shared features between the cognitive and visual domains, for given \hat{V} and \hat{R}, a common representation for the cognitive and visual domain modalities is derived as follows:

$${V}_{ℊ} = {G}_{( V , R )} ( \hat{V} ; {θ}^{ℊ} ) ;$$

$${R}_{ℊ} = {G}_{( V R )} ( \hat{R} ; {θ}^{ℊ} ) ;$$

where G(V, R)(⋅) is an encoding function based on a simple fully connected neural layer, and [symbol] is a shared parameter in the cognitive and visual domains;
a private representation is output by the private channel of the cognitive and visual domains, to capture private information related to the cognitive and visual domains, for given \hat{V} and \hat{R}, the private representation is derived as follows:

$${V}_{s} = {S}_{V} ( \hat{V} ; {θ}_{V}^{s} ) ;$$

$${R}_{s} = {S}_{R} ( \hat{R} ; {θ}_{R}^{s} ) ;$$

where S_{V}(⋅) is a private encoding function of the private channel in the visual domain, S_{R}(⋅) is a private encoding function of the private channel in the cognitive domain, S_{V}(⋅) and S_{R}(⋅) are achieved by the fully connected neural layer,

$${θ}_{s}^{V}$$

is a parameter of the private encoding function of the private channel in the visual domain, and

$${θ}_{s}^{R}$$

is a parameter of the private encoding function of the private channel in the cognitive domain;
S213, after acquiring the common representation and the private representation, performing a concatenation operation on the acquired common representation and the private representation, and performing emotion recognition tasks;
wherein the concatenated representations of the common representation and the private representation of the visual domain and the cognitive domain are respectively defined as:

$$= [ , ⊕ {V}_{s} ] ;$$

$$= [ , ⊕ {R}_{s} ] ;$$

where [symbol] is a concatenated representation of the visual domain, and [symbol] is a concatenated representation of the cognitive domain;
the expressions of the emotion recognition tasks in the visual domain and the cognitive domain are as follows:

$${y}_{V} = BV ( ) ;$$

$${y}_{R} = BR ( ) ;$$

where y_{V} and y_{R} are predicted labels of the images and the EEG signals, respectively, with a K-Nearest Neighbors (KNN) classifier employed as a model for a function B(⋅).

In some embodiments, in S2, the specific steps of the emotion recognition of the learner's dialogue text based on the second-order interactive attention mechanism are as follows:
S221, constructing an explicit information pair and an implicit information pair as inputs for an utterance of learner's learning emotion and learning feeling reports, and sending these pairs to an information association module;
S222, encoding dialogue information, treating the dialogue speech and situation between the learner and the agent as explicit information, treating an inference knowledge as implicit information, and processing the explicit information and the implicit information to obtain a semantic representation of a dialogue speech representation, a situation representation and an inference knowledge;
S223, adopting the information association module to capture significant association words within the explicit information and implicit information, and performing the operations of constructing an association matrix, applying a first-order interactive attention, applying a second-order interactive attention, and storing the association words into memory to obtain an association representation, wherein the expression is as follows: [symbol] ₙ=Encₙ(E_{combine})
where [symbol] ₙ is an association representation, E_{combine} is association information, E_{combine}∈ [symbol] ^{L}^{ₙ}^{×d}, Lₙ is a number of association words in the memory, and Encₙ is an encoder;
S224, inputting the dialogue speech representation, the situation representation, the association representation, and the inference knowledge representation into an aggregation network, acquiring an affective representation, and using the affective representation to predict emotional probabilities, respectively; wherein the expressions for the emotional probabilities are:

$${𝒫}_{d} = ϕ ( A {N}_{f} ( {ℋ}_{d} ) ) ;$$

$${𝒫}_{q} = ϕ ( A {N}_{f} ( {ℋ}_{q} ) ) ;$$

$${𝒫}_{a} = ϕ ( A {N}_{a} ( {ℋ}_{n} ) ) ;$$

$${𝒫}_{tz} = ϕ ( A {N}_{tz} ( {\tilde{ℋ}}_{tz} ) ) ;$$

where, [symbol] _{d}, [symbol] _{q}∈ [symbol] ^{d}^{ₑ}, [symbol] ₐ, [symbol] ₜₓ∈ [symbol] ^{d}^{ₑ}, [symbol] _{d} is an emotional probability of dialogue speech representation, [symbol] _{q} is an the emotional probability of situation representation, [symbol] ₐ is an emotional probability of association representation, [symbol] _{tz} is the emotional probability of inference knowledge, φ is a softmax function, dₑ is a number of emotions, ANₐ and AN_{tz} denote aggregation networks with a same structure but different parameters, [symbol] _{d} is a dialogue speech representation, [symbol] _{q} is a situation representation, [symbol] ₙ is an association representation, [symbol] _{tz} is an inference knowledge representation;
S225, obtaining a final emotional probability by multiplying the respective emotional probabilities, and employing a log-likelihood loss function to optimize the parameters based on the emotional probability and a ground-truth label aff*; wherein the expression is: [symbol] _{aff}= [symbol] _{d}(aff*)· [symbol] _{q}(aff*)· [symbol] ₐ(aff*)· [symbol] _{tz}(aff*); [symbol] _{aff}=−log( [symbol] _{aff}); where [symbol] _{aff} denotes a loss function that trains and optimizes the model by calculating a logarithmic difference between the predicted emotional probability and the true emotional label.

In some embodiments, in S2, the specific steps of the emotional state discrimination of the learner based on the fusion of brain-vision-language multimodal data are as follows:
S231, receiving an emotional prediction result [symbol] _{aff} derived from the second-order attention mechanism, an emotional prediction label y_{V} based on the visual images, and an emotional prediction label y_{R} based on the EEG signals, and concatenating them into a vector X=concat( [symbol] _{aff}, y_{V}, y_{R});
S232, processing the concatenated input X through a multi-layer perceptron (MLP) including multiple fully connected layers, wherein each layer transforms the input via a weight matrix and an activation function to generate the final emotional prediction result, the expression is: [symbol] =MLP(concat( [symbol] _{aff},y_{V},y_{R})), where the MLP is a multi-layer perceptron consisting of multiple fully connected layers and activation functions.

In some embodiments, in S3, the multimodal empathetic response generation framework introduces three models: a perception and retrieval model (PRM), a generation model (GM), and a retrieval augmentation model (RAM), for processing multimodal data; the multimodal empathetic response generation framework employs ViT-G/14, Q-Former, and a linear layer to encode images, utilizes a DALL⋅E2 image decoder to decode images, and utilizes a GPT-4 for language modeling.

In some embodiments, in S3, the specific steps of constructing the empathetic response model based on the multimodal representation are as follows:
S31, multimodal input perception and feature fusion: using a dialogue content between the learner and the agent as multimodal input and converting it into an embedding vector suitable for the large language model processing, encoding images via a pre-trained visual encoder to convert them into an embedding vector eᵥ∈ [symbol] ^{32×d} and fusing with text features through a bootstrapping language image pre-training (BLIP) and the MLP; wherein each text token is embedded as a semantic information vector eₜ∈ [symbol] ^{1×d} for one text.
S32, multimodal output and generation: performing operations of a vocabulary expansion, a text generation, and an image generation to obtain a loss function optimization model, and performing a retrieval and enhanced image generation operation.

In some embodiments, in S32, the expressions of each loss function are:

$${ℒ}_{lm} = - {\sum }_{i = 1}^{k} log p ( {δ}_{i} \vert ζ , {δ}_{1} , … , {δ}_{i - 1} ; θ , Em {b}_{i m g} ) ;$$

$${ℒ}_{p r m} = \frac{1}{2} ( {ℒ}_{txt \to img} + {ℒ}_{img \to txt} ) ;$$

$${ℒ}_{g m} = {‖ {θ}_{Mapper} ( {h}_{gm} , q ) - β ( des , [ a ] ) ‖}_{2}^{2} ;$$

where [symbol] ₗₘ is a language modeling loss; [symbol] _{gm} is an image generation loss; [symbol] ₚᵣₘ is an image retrieval loss;

$$ζ = \{ {e}_{m}^{( 1 )} , {e}_{m}^{( 2 )} , … , {e}_{m}^{( l )} \}$$

and m∈{text, visual} are features of the input text and images; Token={δ₁, . . . δₖ} is a generated text token and visual token; δᵢ∈V*, Emb_{img} is an embedded representation of the image and denotes a visual feature; [symbol] _{txt→img} is a contrastive loss from the text to the image; [symbol] _{img→txt} is a contrastive loss from the image to the text; h_{gm} is a hidden state in the generation task, q is a query feature, β is a frozen text encoder, des is a description of the image, and a is emotional information.

In some embodiments, in S3, the joint learning training of the empathetic response model employs an end-to-end approach to train the model, and uses an adapter fine-tuning method for a joint fine-tuning to synchronously update a limited number of parameters in the LLM, while updating an input linear projection layer and a feature mapping module to obtain a final overall loss function; the final loss function includes the language modeling loss [symbol] ₗₘ, the image generation loss [symbol] _{gm} and the image retrieval loss [symbol] ₚᵣₘ, and the overall loss function is expressed as:

$$ℒ = {λ}_{1} \times {ℒ}_{lm} + {λ}_{2} \times {ℒ}_{gm} + {λ}_{3} \times {ℒ}_{prm} ;$$

where λ₁, λ₂ and λ₃ are hyperparameters, for the perception and retrieval model, [symbol] _{gen}=0; for the generated model, [symbol] ₚᵣₘ=0.

Therefore, the present disclosure proposes a method for agent empathetic response based on brain-machine coupling under the guidance of the learner's emotion, and its beneficial effects are as follows:
(1) The present disclosure proposes a method for agent empathetic response based on brain-machine coupling, which is designed to enhance the learner's empathetic ability by accurately identifying the learner's emotional state and dynamically adjusting the agent's dialogue content based on the learner's emotional state.
(2) The present disclosure integrates EEG signals with multimodal emotion recognition technology to acquire the learner's emotional feedback in real time via brain-machine coupling. On this basis, more empathetic dialogue content is generated, thereby enhancing the learner's emotional cognitive abilities during collaborative learning and promoting the development of their social interaction skills and emotional intelligence.

Further detailed descriptions of the technical scheme of the present disclosure can be found in the accompanying drawings and embodiments.

### BRIEF DESCRIPTION OF THE DRAWINGS

FIG. 1 is a main flow chart of a method for agent empathetic response based on brain-machine coupling under a guidance of a learner's emotion according to the present disclosure;

FIG. 2 is a cognitive-visual learning framework diagram of a method for agent empathetic response based on brain-machine coupling under a guidance of a learner's emotion according to the present disclosure;

FIG. 3 is a diagram of an emotion recognition of a learner's dialogue text based on a second-order interactive attention mechanism of a method for agent empathetic response based on brain-machine coupling under a guidance of a learner's emotion according to the present disclosure;

FIG. 4 is a multimodal empathetic response generation framework diagram of a method for agent empathetic response based on brain-machine coupling under a guidance of a learner's emotion according to the present disclosure.

### DETAILED DESCRIPTION OF THE EMBODIMENTS

In order to make the objectives, the technical solutions, and the advantages of the present disclosure clearer, the following clearly and completely describes the technical solutions in embodiments of the present disclosure with reference to the embodiments of the present disclosure. Apparently, the described embodiments are only some but not all of the embodiments of the present disclosure. All other embodiments obtained by those of ordinary skill in the art based on the embodiments of the present disclosure without involving any creative effort shall fall within the scope of protection of the present disclosure.

Unless otherwise defined, the technical or scientific terms used in the present disclosure shall be those to which the present disclosure belongs.

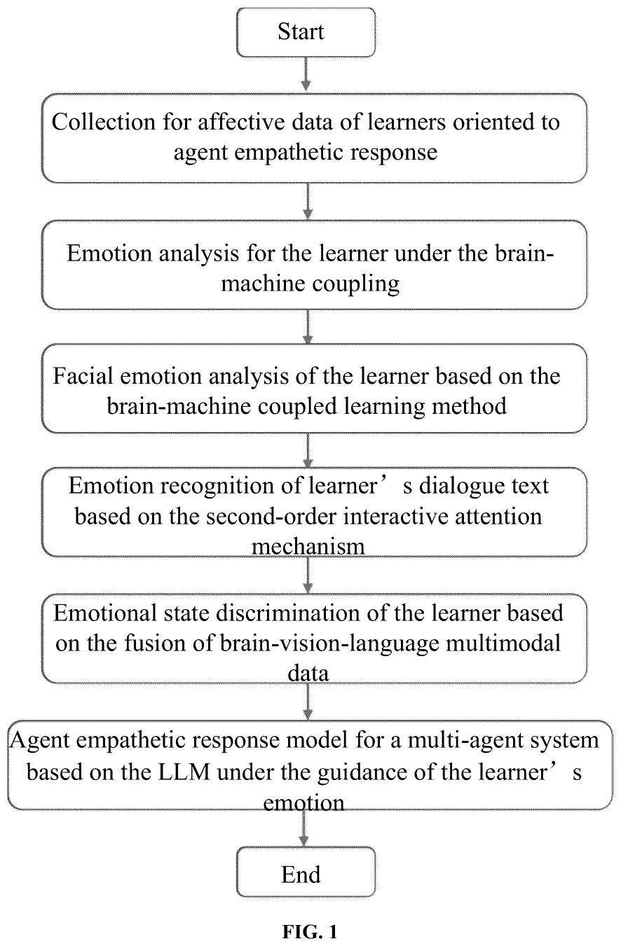

As shown in FIG. 1, the present disclosure proposes a method for agent empathetic response based on brain-machine coupling under the guidance of learner's emotion, and the method includes the following steps:
S1, the multimodal affective data of learners directed to agent empathetic response is collected, in which the collected multimodal affective data includes learner brain data directed to emotional empathy and dialogue data for agent collaboration;
the specific steps of learner brain data collection directed to emotional empathy are as follows:
S11, the learner's brain data is collected in the laboratory environment, allowing the learner to view different emotional images from the data set to acquire the EEG signals; and
S12, the invalid fragments and artifacts are removed from the acquired EEG signals, and the signals within the frequency band of 1 Hz-75 Hz are filtered using the Butterworth filter.

Wherein the EEG signals are collected through the NeuroScan 64 lead EEG cap, including 62 scalp electrodes and 2 reference electrodes, which are placed according to the international 10-20 standard, with a sampling rate of 1 kHz, The images are divided into seven emotional types: happiness, sadness, anger, surprise, fear, disgust and neutrality. Each facial emotional image is displayed for 0.5 s, and there is a 10 s buffer among facial images. In order to reduce interference, irrelevant equipment is turned off during the experiment to ensure a clean environment. The experiment lasted about 13 min.

The specific steps of dialogue data collection for agent collaboration are as follows: after completing the phased classroom learning task, the self-perception of the learning content and the learning emotion are performed by the learner, and reported to the agent, the dialogue and the communication with the agent are generated, and the context data set of the dialogue is recorded as J=[F₁, . . . , Fᵢ, . . . , F_{N}], the dialogue situation is recorded as

$$Q = [ {w}_{1}^{q} , {w}_{2}^{q} , … , {w}_{n}^{q} ] ,$$

and enabling the agent model to understand the emotion A within the dialogue; where,

$${F}_{i} = [ {w}_{1}^{i} , {w}_{2}^{i} , … , {w}_{{n}_{i}}^{i} ]$$

denotes the i^{th} utterance, containing nᵢ words, and the situation Q denotes the situation description composed of n words, where w^{q} is situation background information, which helps to understand the context of the dialogue and generate appropriate responses accordingly.
S2, the emotion analysis is performed on the collected multimodal affective data of the learner under the brain-machine coupling by employing the affective state discrimination method of the multimodal data, specifically the emotion analysis is performed from three parts: the facial emotion analysis of the learner based on the brain-machine coupled learning method, the emotion recognition of the learner's dialogue text based on the second-order interactive attention mechanism, and the emotional state discrimination of the learner based on the fusion of brain-vision-language multimodal data;
the specific steps of the facial emotion analysis of the learner based on the brain-machine coupled learning method are as follows:
S211, extraction of preliminary representations from facial expression images and EEG signals, the preliminary feature extraction is performed on facial expression images and EEG signals by using the EEGNet and the CNNNet;
herein for facial expression images in the visual domain, the improved CNNNet is employed to extract preliminary features, with the preliminary representation in the visual domain denoted as \hat{V}:

$$\hat{V} = 𝒢 V ( V ) ;$$

where [symbol] V is the improved CNNNet, V is the given visual image, and \hat{V} is the preliminary representation of the visual domain;
for the EEG signals, the feature extractor of the EEGNet is employed for the preliminary feature extraction, with the preliminary representation in the cognitive domain denoted as \hat{R}.

$$\hat{R} = 𝒢 R ( R ) ;$$

where [symbol] R is the compact convolutional neural network designed by EEGNet for the EEG brain-machine interface, R is the given EEG signal, and \hat{R} is the preliminary representation of the cognitive domain;
wherein EEG signals and facial expression images constitute two distinct data modalities. Specifically, CNNNet consists of three convolutional modules, each consisting of a convolutional layer, a normalization layer, a nonlinear activation layer, and a max-pooling layer. The output of the third convolutional module reflects the learner's facial emotional response, serving as the preliminary representation in the visual domain. For EEG signals in the cognitive domain, EEGNet functions as the feature extractor. EEGNet (GR) is a compact convolutional neural network specifically designed for EEG-based brain-machine interfaces, including a standard convolutional layer, a depthwise convolutional layer, and a separable convolutional layer. The output of the third convolutional module is utilized as the preliminary feature representation in the cognitive domain to capture changes in the learner's EEG activity.
S212, the cognitive-visual learning framework is constructed, the acquired preliminary representation is projected to the common channel and the private channel, and the common channel and the private channel representations are encoded;
wherein the common channel employs the encoding function G(V, R)(⋅) with shared parameters to learn and capture common information between the cognitive and visual domains, after training, the common channel is configured to extract the shared features between the cognitive and visual domains, for given the \hat{V} and \hat{R}, the common representation for the cognitive and visual domain modalities is derived as follows:

$$= {G}_{( V , R )} ( \hat{V} ; ) ;$$

$$= {G}_{( V , R )} ( \hat{R} ; ) ;$$

where G(V, R)(⋅) is the encoding function based on the simple fully connected neural layer, and [symbol] is the shared parameter in the cognitive and visual domains;
a private representation is output by the private channel of the cognitive and visual domains, to capture private information related to the cognitive and visual domains, for given \hat{V} and \hat{R}, the private representation is derived as follows:

$${V}_{s} = {S}_{V} ( \hat{V} ; {θ}_{V}^{s} ) ;$$

$${R}_{s} = {S}_{R} ( \hat{R} ; {θ}_{R}^{s} ) ;$$

where S_{V}(⋅) is the private encoding function of the private channel in the visual domain, S_{R}(⋅) is the private encoding function of the private channel in the cognitive domain, S_{V}(⋅) and S_{R}(⋅) are achieved by the fully connected neural layer,

$${θ}_{s}^{V}$$

is the parameter of the private encoding function of the private channel in the visual domain, and

$${θ}_{s}^{R}$$

is the parameter of the private encoding function of the private channel in the cognitive domain;

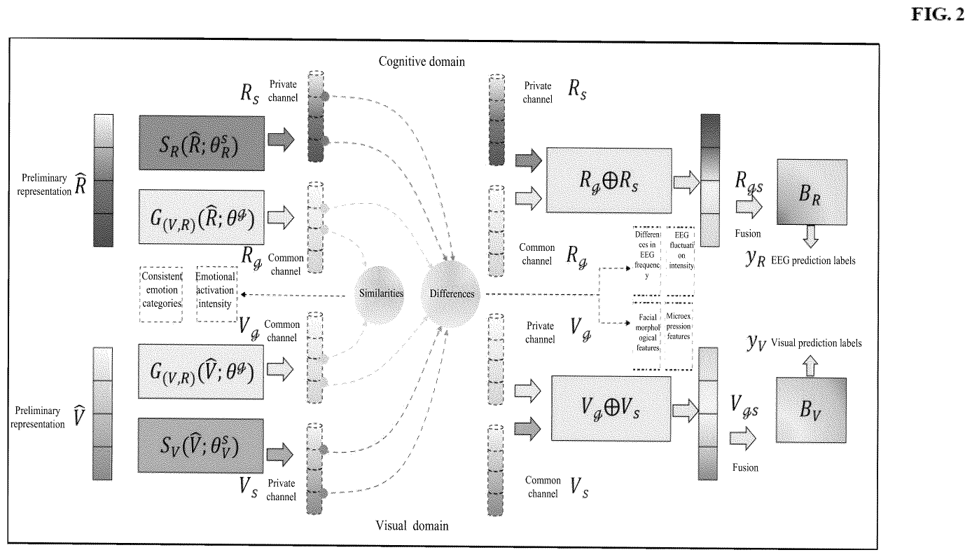

As shown in FIG. 2, the cognitive domain and the visual domain each have their own independent private channels, while the shared commonalities between them are extracted through the common channel. The common features in the cognitive-visual domain refer to emotional response patterns (such as emotional activation intensity and emotional category consistency) that can be shared with EEG signals by observing facial expression images. Private features in the cognitive domain refer to unique EEG, reflecting the way of the brain processes emotions and individual differences (differences in EEG frequency bands, and differences in EEG response intensity, etc.). Private features in the visual domain refer to the distinctive individual variations and details of facial expressions (including facial morphological characteristics and micro-expression features).
S213, after acquiring the common representation and the private representation, the concatenation operation is performed on the acquired common representation and the private representation, and the emotion recognition tasks are performed;
wherein the concatenated representations of the common representation and the private representation of the visual domain and the cognitive domain are respectively defined as:

$$= [ ⊕ {V}_{s} ] ;$$

$$= [ ⊕ {R}_{s} ] ;$$

where [symbol] is the concatenated representation of the visual domain, and [symbol] is the concatenated representation of the cognitive domain;
expressions of the emotion recognition tasks in the visual domain and the cognitive domain are as follows:

$${y}_{V} = BV ( ) ;$$

$${y}_{R} = BR ( ) ;$$

where y_{V} and y_{R} are predicted labels of the images and the EEG signals, respectively, with the KNN classifier employed as the model for the function B(⋅).

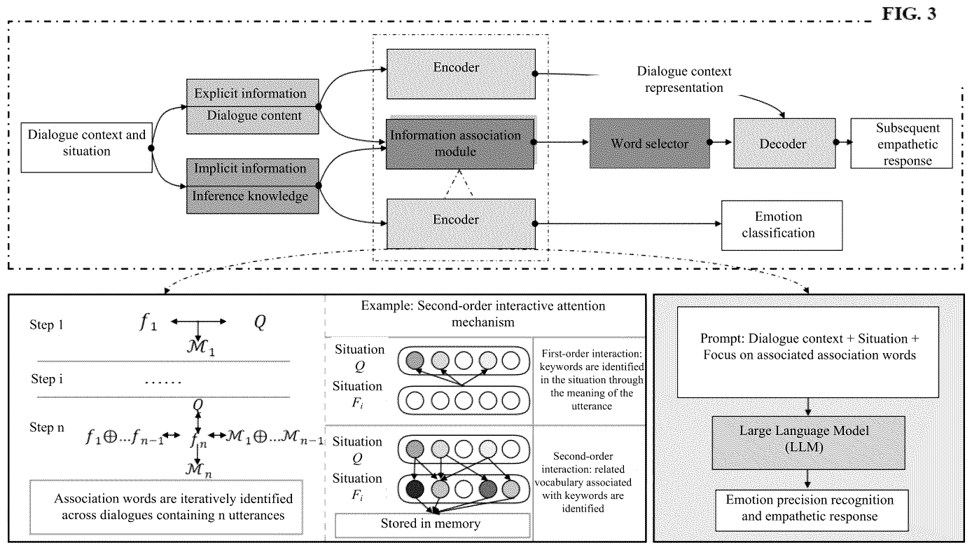

As shown in FIG. 3, the emotion recognition of the learner's dialogue text based on the second-order interactive attention mechanism is proposed. This model utilizes the second-order interactive attention mechanism to analyze both explicit and implicit connotations within dialogue texts in a real classroom setting, thereby improving the accuracy of learner emotion recognition. In this process, after completing phased learning tasks, learners perform self-perception of the learning content and their own learning emotions, subsequently reporting these to the agent via text or emoticon images for emotional communication and feedback. The conversational content is treated as explicit information, while the implicit information includes the learner's emotional state and situational reasoning. Based on this, the dialogue content is encoded from the explicit information and the implicit information.

The specific steps of the emotion recognition of the learner's dialogue text based on the second-order interactive attention mechanism are as follows:
S221, the explicit information pair and the implicit information pair are constructed as inputs for the utterance of the learner's learning emotion and the learning feeling reports, and these pairs are sent to the information association module to understand the learner's current utterance Fᵢ for the learning emotion and learning feeling report.

The constructed explicit information pair is

$$( {E}_{w}^{i} , {E}_{s} ) , ( {E}_{w}^{i} , {E}_{w}^{1} ⊕ … , {E}_{w}^{i - 1} ) , ( {E}_{w}^{i} , {ℳ}_{1} ⊕ … , {ℳ}_{i - 1} ) ;$$

The constructed implicit information pair is:

$$( {ℋ}_{{F}_{i}}^{fq} , {ℋ}_{q}^{fq} ) , ( {ℋ}_{{F}_{i}}^{fq} , {ℋ}_{{F}_{1}}^{fq} ⊕ … , {ℋ}_{{F}_{i - 1}}^{fq} ) , ( {ℋ}_{{F}_{i}}^{fq} , {\overline{ℳ}}_{1} ⊕ … , {\overline{ℳ}}_{i - 1} ) ;$$

where [symbol] ᵢ, and [symbol] ᵢ denote memory, which is used to store associated words and initializes them as empty; when Fᵢ=F₁, the dialogue and memory are empty;
wherein the explicit information pair is used to capture the important associations among the current utterance and situation, dialogue history and memory. The implicit information pair is used to capture the implicit and important associations among the current utterance and situation, dialogue history and memory.
S222, the dialogue information is encoded, the dialogue speech and situation between the learner and the agent are treated as explicit information, the inference knowledge is treated as implicit information, and the explicit information and the implicit information are processed to obtain the semantic representation of the dialogue speech representation, the situation representation and the inference knowledge;
where, the processing process of the explicit information is as follows: the special start tag [CLS] is added before the dialogue speech and situation, respectively, to obtain the utterance Fᵢ and the situation description Q; each round of the utterance Fᵢ and the situation description Q are encoded; the utterance-level encoder Enc_{Utter} is employed for the utterance Fᵢ to generate the sentence representation:

$${ℋ}_{f}^{i} = {Enc}_{Utter} ( {I}_{w}^{i} + {I}_{l} + {I}_{z} ) ;$$

where

$${I}_{w}^{i}$$

is the word embedding, Iₗ is the position embedding, and I_{z} is the state embedding, the state embedding is used to distinguish the speaker (learner or agent) from the listener (agent or learner);
the entire dialogue J is encoded by employing the dialogue-level encoder Enc_{dialogue}:

$${ℋ}_{d} = {Enc}_{dialogue} ( {ℋ}_{f}^{1} ⊕ … ⊕ {ℋ}_{f}^{N} ) ;$$

where,

$${ℋ}_{f}^{i} ∈ {ℝ}^{{n}_{i} \times g} , and {ℋ}_{d} ∈ {ℝ}^{{N}_{J} \times g} ,$$

where nᵢ and [symbol] _{d} denote the length of the i^{th} utterance and the total number of dialogue speeches, respectively, g denotes the hidden layer size, and ⊕ denotes the concatenation operation;
for the situation description Q, the encoder Encₛᵢₜᵤₐₜᵢₒₙ is employed to learn the situation representation:

$${ℋ}_{q} = {Enc}_{situation} ( {I}_{w}^{situation} + {I}_{l}^{situation} ) ;$$

where

$${I}_{w}^{situation} and {I}_{l}^{situation}$$

denote the word embedding and the position embedding of the situation, respectively, [symbol] _{q}∈ [symbol] ^{n×g}, and n is the number of words contained in the situation;
the processing process of implicit information is as follows: inference knowledge [symbol] _{f}_{ᵢ} and [symbol] _{q} are generated via the COMET model; the tag [CLS] is added preceding the inference knowledge, and subsequently the inference knowledge is encoded; the inference knowledge [symbol] _{tz} includes five types of texts: the event effect uEffect, the personal emotional reaction uReact, the personal intention uIntent, the personal need uNeed, and the personal desire uWant; the expression after processing the implicit information is as follows:

$${ℋ}_{tz}^{type} = {Enc}_{dq} ( [ CLS ] ⊕ {𝒦}_{tz}^{type} ) ;$$

where

$${ℋ}_{tz}^{type}$$

denotes the semantic representation of the inference knowledge, tz∈(Fᵢ, q) is the inference knowledge for the i^{th} round of utterance Fᵢ and situation q;
type∈[uEffect, uReact, uIntent, uNeed, uWant], and ⊕ denotes the concatenation operation;
S223, the information association module is adopted to capture significant association words within the explicit information and implicit information, and the operations of constructing the association matrix are performed, the first-order interactive attention is applied, the second-order interactive attention is applied, and the association words are stored into memory to obtain the association representation, so as to understand the dialogue between learner and agent more coherently and comprehensively.

Since association words exist simultaneously in two sentences (the utterance and the situation), the bidirectional selection process for these association words in the two sentences is performed. The specific steps of constructing the association matrix are as follows:
the associated words in the situation (i.e., f→q) are selected based on the utterance. Then, the associated words in the utterance (i.e., q→f) are selected based on the situation. To capture abundant features, a multi-head association matrix is constructed, where D^{q→f} denotes the association score from the situation words to the utterance words, and D^{f→q} denotes the association score from the utterance words to the situation words. The expression of the multi-head association matrix is:

$${D}_{ij}^{q \to f} = {\prod }_{n = 1}^{H} σ ( {( {ω}_{query}^{μ} {τ}_{q , i}^{μ} )}^{T} ( {ω}_{key}^{μ} {τ}_{f , j}^{μ} ) ) ;$$

$${D}_{ij}^{f \to q} = {\prod }_{n = 1}^{H} σ ( {( {ω}_{query}^{μ} {τ}_{f , i}^{μ} )}^{T} ( {ω}_{key}^{μ} {τ}_{q , j}^{μ} ) )$$

where D^{q→f}∈ [symbol] ^{H×g}^{_{q}}^{×g}^{_{f}}, D^{f→q}∈ [symbol] ^{H×g}^{_{f}}^{×g}^{_{q}}, σ is the Sigmoid function, H is the number of multi-heads, and μ denotes the μ^{th} head index in the multi-head attention mechanism. ω, and τ denote the weight matrix and word vector used in the multi-head attention mechanism.

The steps for performing the first-order interactive attention are as follows:
keywords in situations are identified, and situation words with high correlation with utterance words are selected as keywords:

$${D}^{q} = Mean ( {D}^{f \to q} ) ;$$

$${D}_{{k}_{1}}^{q} = ( {D}^{q} ) ;$$

where Mean denotes the mean function, [symbol] denotes the filtering function, k₁ denotes the number of selected keywords, and

$${D}^{q} ∈ {ℝ}^{H \times {g}_{q}} , {D}_{{k}_{1}}^{q} ∈ {ℝ}^{H \times {k}_{1}} .$$

The steps for performing the second-order interactive attention are as follows:
the utterance words having the highest association with key situation words are selected as important association words:

$${D}_{{k}_{2}}^{q \to f} , {Score}_{{k}_{2}}^{q \to f} = {Top}_{{k}_{2}} ( {D}^{f \to q} ) ;$$

where

$${D}_{{k}_{2}}^{q \to f} and {Score}_{{k}_{2}}^{q \to f}$$

are the representation and score of the utterance words associated with the situation words, respectively. k₂ denotes the number of association words.

The steps for performing the storage association memory are as follows:

For explicit information, the keywords

$$( {C}_{fq}^{ek} )$$

between utterances and situations, the association words

$$( {C}_{fh}^{ek} )$$

between utterances and dialogue history, and the association words

$$( {C}_{fm}^{ek} )$$

between utterances and memory are selected:

$${E}_{ek} = {C}_{fq}^{ek} ⊕ {C}_{fh}^{ek} ⊕ {C}_{fm}^{ek} ;$$

the implicit memory is:

$${E}_{i k} = {C}_{fq}^{i k} ⊕ {C}_{fh}^{i k} ⊕ {C}_{fm}^{i k} ;$$

when iterating to the final sentence, the explicit information memory Eₑₖ and implicit information memory Eᵢₖ are combined to form the final association information E_{combine}:

$${E}_{c o m b i n e} = {V}_{e k} ⊕ {V}_{i k} ;$$

where

$${C}_{fq}^{e k} , {C}_{fh}^{e k} , {C}_{fm}^{e k} ∈ {ℝ}^{( 2 H \times {k}_{1} ) \times ( {k}_{2} \times {d}_{h} )} , and {C}_{fq}^{i k} , {C}_{fh}^{i k} , {C}_{fm}^{i k} ∈ {ℝ}^{( 2 H \times {k}_{1} ) \times ( {k}_{2} \times {d}_{h} )} .$$

Based on the memory of association words, the association information is learned through the encoder, and the expression of the association information is:

$${ℋ}_{n} = E n {c}_{n} ( {E}_{c o m b i n e} ) ;$$

where [symbol] ₙ is the association representation, E_{combine} is the association information, E_{combine}∈ [symbol] ^{L}^{ₙ}^{×d}, Lₙ is the number of association words in the memory, and Encₙ is the encoder.
S224, the dialogue speech representation, the situation representation, the association representation, and the inference knowledge representation are input into the aggregation network to acquire the affective representation, and the affective representation is used to predict the emotional probabilities, respectively; wherein the expressions for the emotional probabilities are:

$${𝒫}_{d} = ϕ ( {AN}_{f} ( {ℋ}_{d} ) ) ;$$

$${𝒫}_{q} = ϕ ( {AN}_{f} ( {ℋ}_{q} ) ) ;$$

$${𝒫}_{a} = ϕ ( {AN}_{a} ( {ℋ}_{n} ) ) ;$$

$${𝒫}_{tz} = ϕ ( {AN}_{tz} ( {\tilde{ℋ}}_{tz} ) ) ;$$

where, [symbol] _{d}, [symbol] _{q}∈ [symbol] ^{d}^{ₑ}, [symbol] ₐ, [symbol] _{tz}∈ [symbol] ^{d}^{ₑ}, [symbol] _{d} is the emotional probability of dialogue speech representation, [symbol] _{q} is the the emotional probability of situation representation, [symbol] ₐ is the emotional probability of association representation, [symbol] _{tz} is the emotional probability of inference knowledge, φ is the softmax function, dₑ is the number of emotions, ANₐ and AN_{tz} denote aggregation networks with the same structure but different parameters, [symbol] _{d} is the dialogue speech representation, [symbol] _{q} is the situation representation, [symbol] ₙ is the association representation, [symbol] _{tz} is the inference knowledge representation.
S225, the final emotional probability is obtained by multiplying the emotional probabilities, and the log-likelihood loss function is employed to optimize the parameters based on the emotional probability and the ground-truth label aff*; wherein the expression is: [symbol] _{aff}= [symbol] _{d}(aff*)· [symbol] _{q}(aff*)· [symbol] ₐ(aff*)· [symbol] _{tz}(aff*); [symbol] _{aff}=−log( [symbol] _{aff}); where [symbol] _{aff} denotes the loss function that trains and optimizes the model by calculating the logarithmic difference between the predicted emotional probability and the true emotional label.

The specific steps of the emotional state discrimination of the learner based on the fusion of brain-vision-language multimodal data are as follows:
S231, the emotional prediction result [symbol] _{aff} derived from the second-order attention mechanism, the emotional prediction label y_{V} based on the visual images, and the emotional prediction label y_{R} based on the EEG signals are received, and they are concatenated into the vector X=concat( [symbol] _{aff},y_{V},y_{R});
S232, the concatenated input X is processed through the MLP, including multiple fully connected layers, wherein each layer transforms the input via the weight matrix and the activation function to generate the final emotional prediction result, the expression is: [symbol] =MLP(concat( [symbol] _{aff},y_{V},y_{R})), where the MLP is the multi-layer perceptron consisting of multiple fully connected layers and activation functions.
S3, the multimodal empathetic response generation framework is designed based on results of emotion recognition and the dialogue content, then the empathetic response model is constructed based on the multimodal representation, and the joint learning training of the empathetic response model is performed, thereby forming the agent empathetic response model for the multi-agent system based on the LLM under the guidance of the learner's emotion.

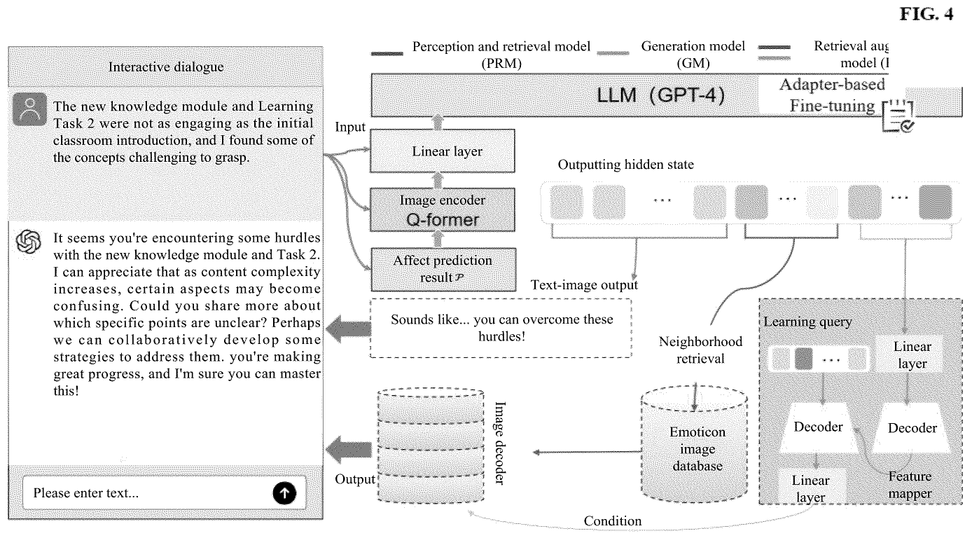

As shown in FIG. 4, the multimodal empathetic response generation framework possesses the capability to perceive learner's emotions based on their EEG and text-based emotional recognition results, as well as dialogue content (including text inputs and emoji inputs), and generate corresponding text replies and emoji responses. Through distinct image generation strategies, this framework derives three models: the perception and retrieval model (PRM), the generation model (GM), and the retrieval augmentation model (RAM). The multimodal empathetic response generation framework employs ViT-G/14, Q-Former, and the linear layer to encode images, utilizes GPT-4 for language modeling, and adopts DALL⋅E2 as the image decoder.

The specific steps of constructing the empathetic response model based on the multimodal representation are as follows:
S31, multimodal input perception and feature fusion: the dialogue content between the learner and the agent is used as multimodal input and converted into the embedding vector suitable for the large language model processing, images are encoded via the pre-trained visual encoder to convert them into the embedding vector eᵥ∈ [symbol] ^{32×d} and fused with text features through the BLIP and the MLP; where each text token is embedded as the semantic information vector eₜ∈ [symbol] ^{1×d} for one text.
S32, multimodal output and generation: the operations of the vocabulary expansion, the text generation, and the image generation are performed to obtain the loss function optimization model, and the retrieval and enhanced image generation operation is performed.

The specific steps of the vocabulary expansion are as follows:
the visual token set V_{img} is incorporated, the visual tokens are divided into two groups, where the first k tokens are used for image retrieval, and the second l-k tokens are used for image generation. The image information is incorporated as part of the generated content, expressed as:

$${V}_{i m g} = \{ [ Image 1 ] , [ Image 2 ] , … , [ Image \{ 𝒵 \} ] ;$$

$${V}_{p r m} = \{ [ Image 1 ] , … , [ I mage \{ k \} ] \} ;$$

$${V}_{g m} = \{ [ I mage \{ k + 1 \} ] , … , [ Image \{ 𝒵 \} ] \} ;$$

where, Vₚᵣₘ is used for the perception and retrieval model and retrieval augmentation model, while V_{gm} is used for the generation model and retrieval augmentation model.

The specific steps for text generation are as follows:
after receiving multimodal input, the joint sequence of text tokens and visual tokens

$${\{ [ Image \{ i \} ] \}}_{i = 1}^{l}$$

is generated. The generated tokens are denoted as

$${\{ [ Image \{ i \} ] \}}_{i = 1}^{l} ,$$

where δᵢ∈V*. The loss function [symbol] ₗₘ is defined as:

$${ℒ}_{lm} = - {\sum }_{i = 1}^{k} log p ( {δ}_{i} \vert ζ , {δ}_{1} , … , {δ}_{i - 1} ; θ , E m {b}_{i m g} ) ;$$

Where

$$ζ = \{ {e}_{m}^{( 1 )} , {e}_{m}^{( 2 )} , … , {e}_{m}^{( l )} \}$$

and m∈{text, visual} are features of the input text and images, Emb_{img} is the embedded representation of the image and denotes the visual feature for alignment with text embeddings, where the model is optimized via a loss function that maximizes the generation probability.

In the image retrieval stage, the agent empathetic response model aligns the hidden state hₚᵣₘ corresponding to Vₚᵣₘ to the retrieval space via contrastive learning and utilizes cosine similarity to measure the similarity of projection vector, yielding the image retrieval loss [symbol] ₚᵣₘ. By combining contrastive losses of the text-to-image (txt→img) and image-to-text (img→txt), the mapping between images and text is optimized. The image retrieval loss [symbol] ₚᵣₘ is:

$${ℒ}_{p r m} = \frac{1}{2} ( {ℒ}_{txt \to img} + {ℒ}_{img \to txCt} )$$

where [symbol] ₚᵣₘ is used to optimize the loss of the retrieval projection layer.

The specific steps for image generation are as follows:
in the image generation stage, the generated image is generated by the DALL·E 2 decoder, and the loss between the generated image and the actual image is minimized.

The loss function [symbol] _{gm} is expressed as:

$${ℒ}_{g m} = {‖ {θ}_{Mapper} ( {h}_{gm} , q ) - β ( d es , [ a ] ) ‖}_{2}^{2} ;$$

where h_{gm} is the hidden state in the generation task, q is the query feature, β is the frozen text encoder, des is the description of the image, and a is the emotional information.

The steps for performing the retrieval and enhanced image generation operation are as follows:
an image is retrieved as a latent representation c_{Img} to enhance the generation process. During image generation, h_{gm} continues to serve as a condition. This approach continues generating on the retrieved image, thereby expanding image diversity and maintaining image quality.

The specific steps of the joint learning training of the empathetic response model are as follows:

The end-to-end approach is employed to train the model, and the adapter fine-tuning method is used for the joint fine-tuning to synchronously update the limited number of parameters in the LLM, while updating the input linear projection layer and the feature mapping module to obtain the final overall loss function; the final loss function includes the language modeling loss [symbol] ₗₘ, the image generation loss [symbol] _{gm} and the image retrieval loss [symbol] ₚᵣₘ, and the overall loss function is expressed as:

$$ℒ = {λ}_{1} \times {ℒ}_{l m} + {λ}_{2} \times {ℒ}_{g m} + {λ}_{3} \times {ℒ}_{p r m} ;$$

where λ₁, λ₂ and λ₃ are hyperparameters, for the perception and retrieval model, [symbol] _{gen}=0; for the generated model, [symbol] ₚᵣₘ=0.

Through this multi-task joint training, the agent empathetic response model is enabled to generate reasonable responses enriched with visual elements in empathy-rich dialogues, thereby enhancing both the diversity and emotional understanding of the agent's empathetic responses and the dialogues.

Therefore, the method for agent empathetic response based on brain-machine coupling under guidance of the learner's emotion is provided by the present disclosure, which is designed to enhance the learner's empathetic ability by accurately identifying the learner's emotional state and dynamically adjusting the agent's dialogue content based on the emotional state. Furthermore, the method integrates EEG signals with multimodal emotion recognition technology to acquire the learner's emotional feedback in real time via brain-machine coupling. On this basis, more empathetic dialogue content is generated, thereby enhancing the learner's emotional cognitive abilities during collaborative learning and promoting the development of their social interaction skills and emotional intelligence.

Finally, it should be noted that the above embodiments are merely used for describing the technical solutions of the present disclosure, rather than limiting the same. Although the present disclosure has been described in detail with reference to the preferred examples, those of ordinary skill in the art should understand that the technical solutions of the present disclosure may still be modified or equivalently replaced. However, these modifications or substitutions should not make the modified technical solutions deviate from the spirit and scope of the technical solutions of the present disclosure.

What is claimed is:

1. A method for agent empathetic response based on brain-machine coupling under a guidance of a learner's emotion, comprising the following steps:
S1, collecting multimodal affective data of learners directed to an agent empathetic response, wherein the collected multimodal affective data comprises learner brain data directed to emotional empathy and dialogue data for agent collaboration;
S2, performing an emotion analysis on the collected multimodal affective data of the learner under a brain-machine coupling by employing an affective state discrimination method on the multimodal data, wherein the emotion analysis is performed in the following three parts: a facial emotion analysis of the learner based on the brain-machine coupled learning method, an emotion recognition of a learner's dialogue text based on a second-order interactive attention mechanism, and an emotional state discrimination of the learner based on a fusion of brain-vision-language multimodal data; and
S3, designing a multimodal empathetic response generation framework based on results of emotion recognition and a dialogue content, then constructing an empathetic response model based on a multimodal representation, and performing a joint learning training of the empathetic response model, thereby forming an agent empathetic response model for a multi-agent system based on a LLM under the guidance of the learner's emotion;
wherein in S3, the specific steps of constructing the empathetic response model based on the multimodal representation are as follows:
S31, multimodal input perception and feature fusion, comprising using the dialogue content between the learner and the agent as a multimodal input and converting it into an embedding vector suitable for the large language model processing, encoding images via a pre-trained visual encoder to convert them into an embedding vector eᵥ∈ [symbol] ^{32×d} and fusing with text features through a BLIP and a MLP; wherein each text token is embedded as a semantic information vector eₜ∈ [symbol] ^{1×d} for one text, where [symbol] is a real vector space, and d is an embedding vector dimension; and
S32, multimodal output and generation, comprising performing operations of a vocabulary expansion, a text generation, and an image generation to obtain a loss function optimization model, and performing a retrieval and enhanced image generation operation.

2. The method for agent empathetic response based on brain-machine coupling under a guidance of a learner's emotion according to claim 1, wherein in S1, the specific steps of learner brain data collection directed to emotional empathy are as follows:
S11, collecting the learner's brain data in a laboratory environment, allowing the learner to view different emotional images from a data set to acquire EEG signals; and
S12, removing invalid fragments and artifacts from the acquired EEG signals, and filtering the signals within a frequency band of 1 Hz-75 Hz using a Butterworth filter.

3. The method for agent empathetic response based on brain-machine coupling under a guidance of a learner's emotion according to claim 1, wherein in S1, the specific steps of dialogue data collection for agent collaboration are as follows: after completing a phased classroom learning task, performing a self-perception of learning content and learning emotion by the learner, reporting the self-perception to the agent, generating a dialogue and a communication with the agent, and recording a context data set of the dialogue as J=[F₁, . . . , Fᵢ, . . . , F_{N}], recording a dialogue situation as Q=
$$[ {w}_{1}^{q} , {w}_{2}^{q} , … , {w}_{n}^{q} ] ,$$
and enabling the agent model to understand an emotion A within the dialogue; where
$${F}_{i} = [ {w}_{1}^{i} , {w}_{2}^{i} , … , {w}_{{n}_{i}}^{i} ]$$
denotes an i^{th} utterance, containing nᵢ words, and the situation Q denotes a situation description composed of n words, where w^{q} is situation background information.

4. The method for agent empathetic response based on brain-machine coupling under a guidance of a learner's emotion according to claim 1, wherein in S2, the specific steps of the facial emotion analysis of the learner based on the brain-machine coupled learning method are as follows:
S211, performing a preliminary feature extraction on facial expression images and EEG signals by using an EEGNet and a CNNNet;
wherein for facial expression images in a visual domain, an improved CNNNet is employed to extract preliminary features, with a preliminary representation in the visual domain denoted as \hat{V}:
$$\hat{V} = 𝒢 V ( V ) ;$$
where [symbol] V is the improved CNNNet, V is a given visual image, and \hat{V} is the preliminary representation of the visual domain;
wherein, for the EEG signals, a feature extractor of the EEGNet is employed for a preliminary feature extraction, with a preliminary representation in a cognitive domain denoted as \hat{R}:
$$\hat{R} = 𝒢 R ( R ) ;$$
where [symbol] R is a compact convolutional neural network designed by EEGNet for an EEG brain-machine interface, R is a given EEG signal, and \hat{R} is the preliminary representation of the cognitive domain;
S212, constructing a cognitive-visual learning framework, projecting the acquired preliminary representation to a common channel and a private channel, and encoding common channel and the private channel representations;
wherein the common channel employs an encoding function G(V, R)(⋅) with shared parameters to learn and capture common information between the cognitive and visual domains; wherein, after training, the common channel is configured to extract the shared features between the cognitive and visual domains, and, for given the \hat{V} and \hat{R}, a common representation for the cognitive and visual domains modalities is derived as follows:
$$\begin{matrix}= {𝒢}_{( V , R )} ( \hat{V} ; ) ; \\ = {𝒢}_{( V , R )} ( \hat{R} ; ) ;\end{matrix}$$
where G(V, R)(⋅) is an encoding function based on a simple fully connected neural layer, and [symbol] is a shared parameter in the cognitive and visual domains;
wherein a private representation is output by the private channel of the cognitive and visual domains, to capture private information related to the cognitive and visual domains, and, for given \hat{V} and \hat{R}, the private representation is derived as follows:
$$\begin{matrix}{V}_{s} = {S}_{V} ( \hat{V} ; {θ}_{V}^{s} ) ; \\ {R}_{s} = {S}_{R} ( \hat{R} ; {θ}_{R}^{s} ) ;\end{matrix}$$
where S_{V}(⋅) is a private encoding function of the private channel in the visual domain, S_{R}(⋅) is a private encoding function of the private channel in the cognitive domain, S_{V}(⋅) and S_{R}(⋅) are achieved by the fully connected neural layer,
$${θ}_{s}^{V}$$
is a parameter of the private encoding function of the private channel in the visual domain, and
$${θ}_{s}^{R}$$
is a parameter of the private encoding function of the private channel in the cognitive domain;
S213, after acquiring the common representation and the private representation, performing a concatenation operation on the acquired common representation and the private representation, and performing emotion recognition tasks;
wherein the concatenated representations of the common representation and the private representation of the visual domain and the cognitive domain are respectively defined as:
$$\begin{matrix}= [ ⊕ {V}_{s} ] ; \\ = [ ⊕ {R}_{s} ] ;\end{matrix}$$
where [symbol] is a concatenated representation of the visual domain, and [symbol] is a concatenated representation of the cognitive domain;
expressions of the emotion recognition tasks in the visual domain and the cognitive domain are as follows:
$$\begin{matrix}{y}_{V} = BV ( ) ; \\ {y}_{R} = BR ( ) ;\end{matrix}$$
where y_{V} and y_{R} are predicted labels of the images and the EEG signals, respectively, with a KNN classifier employed as a model for a function B(⋅), B_{V}(⋅) is a decoding function of the emotion recognition task in the visual domain, and B_{R}(⋅) is a decoding function of the emotion recognition task in the cognitive domain.

5. The method for agent empathetic response based on brain-machine coupling under a guidance of a learner's emotion according to claim 1, wherein in S2, the specific steps of the emotion recognition of the learner's dialogue text based on the second-order interactive attention mechanism are as follows:
S221, constructing an explicit information pair and an implicit information pair as inputs for an utterance of a learner's learning emotion and learning feeling reports, and sending these pairs to an information association module;
S222, encoding dialogue information, treating the dialogue speech and situation between the learner and the agent as explicit information, treating an inference knowledge as implicit information, and processing the explicit information and the implicit information to obtain a semantic representation of a dialogue speech representation, a situation representation, and an inference knowledge;
S223, adopting the information association module to capture significant association words within the explicit information and implicit information, and performing the operations of constructing an association matrix, applying a first-order interactive attention, applying a second-order interactive attention, and storing the association words into memory to obtain an association representation, wherein the expression is as follows:
$${ℋ}_{n} = E n {c}_{n} ( {E}_{c o m b i n e} )$$
where [symbol] ₙ is an association representation, E_{combine} is association information, E_{combine}∈ [symbol] ^{L}^{ₙ}^{×d}, [symbol] is a real vector space, and d is an embedding vector dimension, Lₙ is a number of association words in the memory, and Encₙ is an encoder;
S224, inputting the dialogue speech representation, the situation representation, the association representation, and the inference knowledge representation into an aggregation network, acquiring an affective representation, and using the affective representation to predict emotional probabilities, respectively; wherein the expressions for the emotional probabilities are:
$$\begin{matrix}{𝒫}_{d} = ϕ ( A {N}_{f} ( {ℋ}_{d} ) ) ; \\ {𝒫}_{q} = ϕ ( A {N}_{f} ( {ℋ}_{q} ) ) ; \\ {𝒫}_{a} = ϕ ( A {N}_{a} ( {ℋ}_{n} ) ) ; \\ {𝒫}_{tz} = ϕ ( A {N}_{tz} ( {\tilde{ℋ}}_{tz} ) ) ;\end{matrix}$$
where [symbol] _{d}, [symbol] _{q}∈ [symbol] ^{d}^{ₑ}, [symbol] ₐ, [symbol] _{tz}∈ [symbol] ^{d}^{ₑ}, [symbol] ^{d}^{ₑ} denotes a real vector space of a dimension dₑ, dₑ is a number of emotions, [symbol] _{d} is an emotional probability of dialogue speech representation, [symbol] _{q} is an the emotional probability of situation representation, [symbol] ₐ is an emotional probability of association representation, [symbol] _{tz} is the emotional probability of inference knowledge, φ is a softmax function, AN_{f} is the aggregation network used to deal with related inputs for dialogue speech representation and situation representation, ANₐ and AN_{tz} denote aggregation networks with a same structure but different parameters, wherein ANₐ is used to deal with related inputs for association representation and AN_{tz} is used to deal with related inputs for inference knowledge representation, [symbol] _{d} is a dialogue speech representation, [symbol] _{q} is a situation representation, [symbol] ₙ is an association representation, [symbol] _{tz} is an inference knowledge representation;
S225, obtaining a final emotional probability by multiplying the respective emotional probabilities, and employing a log-likelihood loss function to optimize the parameters based on the emotional probability and a ground-truth label aff*; wherein the expression is: [symbol] _{aff}= [symbol] _{d}(aff*)· [symbol] _{q}(aff*)· [symbol] ₐ(aff*)· [symbol] _{tz}(aff*); [symbol] _{aff}=−log( [symbol] _{aff}); wherein [symbol] _{aff} denotes a loss function that trains and optimizes the model by calculating a logarithmic difference between the predicted emotional probability and the true emotional label.

6. The method for agent empathetic response based on brain-machine coupling under a guidance of a learner's emotion according to claim 1, wherein in S2, the specific steps of the emotional state discrimination of the learner based on the fusion of brain-vision-language multimodal data are as follows:
S231, receiving an emotional prediction result [symbol] _{aff} derived from the second-order attention mechanism, an emotional prediction label y_{V} based on the visual images, and an emotional prediction label y_{R} based on the EEG signals, and concatenating them into a vector X=concat( [symbol] _{aff},y_{V},y_{R});
S232, processing the concatenated input X through the MLP comprising a plurality of fully connected layers, wherein each layer transforms the input via a weight matrix and an activation function to generate the final emotional prediction result according to the expression: [symbol] =MLP(concat( [symbol] _{aff},y_{V},y_{R})), wherein the MLP is a multi-layer perceptron consisting of a plurality of fully connected layers and activation functions.

7. The method for agent empathetic response based on brain-machine coupling under a guidance of a learner's emotion according to claim 1, wherein in S3, the multimodal empathetic response generation framework introduces three models: a perception and retrieval model (PRM), a generation model (GM), and a retrieval augmentation model (RAM), for processing multimodal data; wherein the multimodal empathetic response generation framework employs ViT-G/14, Q-Former, and a linear layer to encode images, utilizes a DALL⋅E2 image decoder to decode images, and utilizes a GPT-4 for language modeling.

8. The method for agent empathetic response based on brain-machine coupling under a guidance of a learner's emotion according to claim 1, wherein in S31, feature expressions of input texts and images subjected to the multimodal input perception and feature fusion process are:
$$\begin{matrix}ζ = \{ {e}_{m}^{( 1 )} , {e}_{m}^{( 2 )} , … , {e}_{m}^{( l )} \} ; \\ m ∈ \{ text , visual \} ;\end{matrix}$$
where ζ is a context embedding sequence, and is provided to the language model for a conditional generation of content,
$${e}_{m}^{( i )}$$
denotes an embedding vector of an i^{th} modal tag, subscript m is a modal indicator, to denote a modal type of embedding, text is text, and image is visual;
wherein, after receiving the multimodal input, a generated joint sequence expression of text tokens and visual tokens is:
$$Token = \{ {δ}_{1} , … , {δ}_{k} \} ;$$
where δᵢ∈V*, δᵢ is an i^{th} generated joint sequence of text tokens and visual tokens, and V* is a mixed lexical space containing text tokens and visual tokens;
wherein in S32, the expressions of each loss function are:
$${ℒ}_{l m} = - {\sum }_{i = 1}^{k} log p ( {δ}_{i} \vert ζ , {δ}_{1} , … , {δ}_{i - 1} ; θ , E m {b}_{i m g} ) ;$$
$${ℒ}_{p r m} = \frac{1}{2} ( {ℒ}_{txt \to img} + {ℒ}_{img \to txt} ) ;$$
$${ℒ}_{g m} = {‖ {θ}_{Mapper} ( {h}_{gm} , q ) - β ( des , [ a ] ) ‖}_{2}^{2} ;$$
where [symbol] ₗₘ is a language modeling loss; [symbol] _{gm} is an image generation loss; [symbol] ₚᵣₘ is an image retrieval loss; Emb_{img} is an embedded representation of the image and denotes a visual feature; [symbol] _{txt→img} is a contrastive loss from the text to the image; [symbol] _{img→txt} is a contrastive loss from the image to the text; h_{gm} is a hidden state in the generation task, q is a query feature, β is a frozen text encoder, des is a description of the image, and a is emotional information.

9. The method for agent empathetic response based on brain-machine coupling under a guidance of a learner's emotion according to claim 1, wherein in S3, the joint learning training of the empathetic response model employs an end-to-end approach to train the model, and uses an adapter fine-tuning method for a joint fine-tuning to synchronously update a limited number of parameters in the LLM, while updating an input linear projection layer and a feature mapping module to obtain a final overall loss function; wherein the final loss function comprises the language modeling loss [symbol] ₗₘ, the image generation loss [symbol] _{gm} and the image retrieval loss [symbol] ₚᵣₘ, and the overall loss function is expressed as:
$$ℒ = {λ}_{1} \times {ℒ}_{l m} + {λ}_{2} \times {ℒ}_{g m} + {λ}_{3} \times {ℒ}_{p r m} ;$$
where λ₁, λ₂ and λ₃ are hyperparameters, for the perception and retrieval model, [symbol] _{gen}=0; for the generated model, [symbol] ₚᵣₘ=0.
